(19) United States
(12) Patent Application Publication
(10) Pub. No.: US 2025/0194951 A1
(43) Pub. Date: Jun. 19, 2025

(54) BODY MAP CREATION DEVICE AND BODY MAP CREATION METHOD

(71) Applicant: RENATO SCIENCE INC., Kyoto (JP)

(72) Inventors: Tatsuhiko ARAFUNE, Tokyo (JP); Yukinori HASEGAWA, Kyoto (JP)

(21) Appl. No.: 18/562,987

(22) PCT Filed: Mar. 28, 2023
(86) PCT No.: PCT/JP2023/012577

§ 371 Date: Mar. 7, 2025

(30) Foreign Application Priority Data
Mar. 29, 2022 (JP) 2022053126

(51) Int. Cl. A61B 5/107; A61B 5/00

(52) U.S. Cl. CPC A61B 5/107; A61B 5/4312; A61B 5/7225

(57) ABSTRACT

The present invention provides a body map to support reconstructive surgery to reproduce a shape together with a touch. A body mapping device 100 according to one embodiment of the present invention comprises an attachment part 30 which is attached tightly to a breast B which is a protruding physical portion of a measured person K, tactile sensors 40 arranged at the attachment part 30, a measurement part 10 which scans a shape of the breast B of the measured person K and acquires three-dimensional coordinate data, a storage part 20 which stores the coordinate data, and a processing part 50 which carries out information processing to associate the coordinate data with haptic data acquired by the tactile sensors 40.

### TECHNICAL FIELD

The present invention relates to a body mapping device for correlating a shape of a protruding physical portion and a dynamic feature with each other and measuring the same.

Specifically, the present invention relates to a body mapping device and a body mapping method in which on the basis of three-dimensional data prior to physical portion surface surgery before excision of a diseased portion and haptic data of a physical portion at a plurality of parts corresponding to the three-dimensional data, quantified quantitative criteria is obtained so as to support optimal reconstructive surgery.

### BACKGROUND ART

In recent years, surgery for excising a diseased portion is performed to save lives, etc. and the average life expectancy is thus likely to further increase. Moreover, to lead a more satisfactory life after excision of a diseased portion, there have been more and more cases in which body reconstruction surgery for reconstructing an excised physical portion is carried out.

Body reconstruction surgery, in particular, breast reconstruction surgery has been on the rise and various tools to be used for such breast reconstruction surgery have been developed. Examples of prior art may include Breast-Rugle by Medic Engineering Corporation (end of sale) as an existing software which deals with breast shapes in three-dimensional images, and softwares belonging to VECTRA series of breast shape measurement devices manufactured by Canfield Scientific Inc. in the United States of America (domestic sales by Integral Corporation).

The inventors provide a body mapping method, a body mapping program, and a recording medium thereof in which, for the purpose of supporting breast reconstruction surgery, on the basis of three-dimensional data prior to physical portion surface surgery before excision of a diseased portion and three-dimensional data posteriori to physical portion surface surgery after excision of the diseased portion, quantified quantitative criteria is obtained so as to enable optimal reconstructive surgery (see patent literature 1).

### PRIOR ART DOCUMENT

### Patent Literature

Patent literature 1: JP6791482B

### SUMMARY OF INVENTION

### Technical Problem

The features disclosed in patent literature 1 are effective in dimensionally reproducing a shape of a physical portion but fall short of reproducing a touch of the reproduced physical portion.

Known are sensors for acquiring shape data according to the features disclosed in patent literature 1 and measuring the hardness and the pressure sensitivity according to developments in robotics technologies, whereas associating shape data which are three-dimensional data and haptic values, such as hardness and pressure sensitivity values, with each other and using these data for reconstructive surgery while checking consistency between such data have been difficult.

In other words, measurement data on the hardness and the pressure sensitivity were effective only at coordinates of parts measured. Moreover, acquiring data such that vectors are vertical to a surface to be measured, i.e., similar to each other and integrating such data with shape data were necessary.

Further, acquired mapping data require abnormal value determination (for example, about a cancer) and information processing and evaluation of changes of shape data due to gravity and accompanying changes of hardness and pressure-sensitivity data, and the like, which cannot be addressed by simple data processing alone.

Thus, from medical knowledge, diagnosis statuses so far, and the like, determination and threshold value setting in analogy to an expert system is necessary, but a feature with such elements has never been disclosed.

The present invention has been made in view of such actual circumstances in the background art and it is an object of the present invention to provide a body map to support reconstructive surgery to reproduce a shape together with a touch.

### Solution to Problem

The present invention is a body mapping device for correlating a shape of a protruding physical portion and a dynamic feature with each other and measuring the same, thereby solving the above problem.

According to one aspect of the present invention, the body mapping device comprises:
a measurement part which measures three-dimensionally a shape of a physical portion;
a storage part which stores coordinate data of the shape acquired by the measurement part;
an attachment part which is attached tightly to the physical portion and provided with a grid having a certain grid spacing;
a plurality of tactile sensors which are arranged at corresponding intersections of the grid of the attachment part and measure the dynamic feature in a direction vertical to a surface of contact between the physical portion and the attachment part; and
a processing part which associates haptic data acquired by the tactile sensors with the coordinate data of the intersections at which the haptic data are acquired, and creates the body map.

This configuration is characterized by coordinate data of the shape of the protruding physical portion and association of these coordinate data with the dynamic feature.

The protruding physical portion includes a “breast”, a “nose”, an “ear”, an “arm”, an “leg”, and the like. Lack of such physical portions due to an injury, a disease, etc. may be followed by reconstructive surgery.

In view of such situations, this configuration provides three-dimensional measurement of an appearance of a part to be reconstructed.

For three-dimensional measurement, for example, the feature disclosed in patent literature 1 may be employed or a so-called “3D body scan” for which a large number of products and services have been provided in recent years may be employed.

Further, according to this configuration, the dynamic feature acquired by the plurality of tactile sensors provided to the attachment part is measured in such a manner as to be superposed over a coordinate data measurement position of a three-dimensional shape of the protruding physical portion, and coordinate data and haptic data which are the dynamic features are associated with each other.

Note that the dynamic feature acquired by the tactile sensors is a representation of a sense produced by a touch of an object with the surface of an animal and provided in response to mechanical contact in terms of a dynamic feature, such as the force, the acceleration, and the pressure.

For the attachment part, an attachment wear similar to a brassiere may be employed when the protruding physical portion is a “breast”. Note that the attachment part desirably has a shape and is made of a material so as to have a property, such as tight attachment, stretchability with the shape retention, and no oppression while maintaining a natural shape in relation to a part on which the attachment part is to be attached.

Note that according to this configuration, the protruding physical portion is partitioned into small regions by means of the attachment part provided with the grid, which facilitates consistency with data acquired by a “3D body scan” or the like and visual determination of a measurement result.

This grid is not limited to having a rectangular shape, such as a square, having an exact dimension and an equal grid spacing but may have a plurality of measurement points suitable for identifying coordinate data with haptic data at the time of body mapping and for providing support at the time of reconstruction.

For example, a finely-partitioned grid may be employed for a portion a shape of which largely changes, a boldly-partitioned grid may be employed for a portion a shape of which slightly changes, or a grid shape may change in accordance with expansion and contraction of a material of the attachment part.

Further, the dynamic feature acquired by the tactile sensors arranged at the attachment part is configured to be in a direction vertical to a surface of contact between the physical portion and the attachment part, thereby matching vectors of the dynamic feature with one another and eliminating a component of force in an oblique direction so that consistency of shape data and the dynamic feature between a plurality of data is ensured.

According to the present invention, regardless of unevenness of a surface of contact to be measured, coordinate data of the protruding physical portion and haptic data which are the dynamic features can be identified with each other at a plurality of measurement points and precisely associated with each other.

In the above configuration, the attachment part may include: a frame which surrounds an outer edge of the physical portion; and a veil a rim of which is fitted to the frame, the veil being made of an elastic material.

According to this configuration, for example, a wire-like frame employed for a brassiere which surrounds the protruding physical portion and, for example, an elastic stretchable resin film (Panasonic Corporation: https://news.panasonic.com/jp/press/data/2015/12/jn151224-2/jn151224-2.html) for the veil fixed to a rim of the frame so as to cover the frame may be used so that more accurate shape data can be assured.

The stretchable resin film described by way of example is made of a soft and flexible film-like insulating material and has an excellent expansibility, which is preferable also for arranging the tactile sensors.

In the above configuration, the haptic data may be force data.

The force data include the force (pressure), the hardness, the magnitude of a torque, the direction, etc. and the tactile sensors detect such force data.

According to the above configuration, coordinate data which are three-dimensional data and the hardness and pressure-sensitivity values which are one-dimensional force data (haptic data) are associated with each other and consistency between such data can be achieved.

Note that for the force data (haptic data), a tactile sensor which includes slip sense data but is flexibly applicable, as appropriate, depending upon applications may be selected.

In the above configuration, the tactile sensors may include a force sensor of an electrical resistance type, a capacitive type, a piezoelectric type or an optical type.

According to the above configuration, a tactile sensor of the following types:
a tactile sensor of an electrical resistance type which detects the magnitude of a force using an object in which an electrical resistance value changes in a certain manner when a force is applied;
a tactile sensor of a capacitive type which detects the magnitude of a force using a configuration in which the capacitance changes in a certain manner due to a force;
a tactile sensor of a piezoelectric type which detects the magnitude of a force using a piezoelectric element in which the voltage is generated when a force is applied; and
a tactile sensor of an optical type in which a part at which a force is to be applied has a pattern printed, the tactile sensor detecting through an optical sensor a change of the pattern due to a force to determine the magnitude of the force may be employed, as appropriate and as necessary, whereby force data useful and independent of environments and conditions can be acquired.

In the above configuration, the processing part may include a criteria map which indicates a relationship between coordinate data of the physical portion of a healthy body and haptic data having a statistically predetermined range in advance, and transmit a signal when measured haptic data do not fall within the predetermined range of the criteria map.

According to the above configuration, statistically processing in advance coordinate data of the physical portion of a healthy body and haptic data and setting a certain range or a threshold value for a potentially heathy body, thereby allowing for diagnosis support for abnormal value determination (for example, about a cancer) for the protruding physical portion.

Moreover, from results of machine learning, deep learning, etc. using such statistically processed data group, medical knowledge of specialist doctors, diagnosis statuses so far, and the like, determination and threshold value setting in analogy to an expert system can be made and the accuracy of diagnosis support can be improved.

Further, according to the above configuration, selection of a material used for reconstruction of a physical portion, a manufacturing process, etc. can be supported.

In addition, statistically processed data can be used for data for validation when a structural model for a reconstructed part based on a finite element method or a boundary element method is constructed.

Sample products using such selected material, manufacturing process, etc. and structural models can be used for a simulation of a physical portion posteriori to reconstructive surgery, and are thus a reliable material for a patient to undergo surgery.

In the above configuration, the physical portion may be a breast.

Body reconstruction surgery, in particular, breast reconstruction surgery has been on the rise, and according to the above configuration, the reproducibility of reconstruction during breast reconstruction surgery can be improved.

In the above configuration, the attachment part may be attached to a breast reconstructed after breast excision by surgical excision of a diseased portion to measure the reconstructed breast on the basis of the body map as of presurgery.

According to the above configuration, the completeness of a reconstructed breast can be grasped not only by means of a sense of touch of a patient herself or a doctor but also as specific numerical values, whereby a condition after surgery can be evaluated in a more objective manner.

The present invention is a body mapping method for correlating a shape of a protruding physical portion and a dynamic feature with each other and measuring the same.

According to one aspect of the present invention, the body mapping method may comprise: a step of measuring three-dimensionally a shape of the physical portion; a step of storing data of the shape acquired by the measurement part; a step of attaching an attachment part which is attached tightly to the physical portion and provided with a grid having a certain grid spacing; and a step of creating the body map by associating data acquired by a plurality of tactile sensors which are arranged at corresponding intersections of the grid of the attachment part and measure the dynamic feature in a direction vertical to a surface of contact between the physical portion and the attachment part with the intersections at which the data are acquired.

The present invention can provide the method in which, regardless of unevenness of a surface of contact to be measured, coordinate data of the protruding physical portion and haptic data which are the dynamic features can be identified with each other at a plurality of measurement points and precisely associated with each other.

Note that the present invention may be configured as a program in accordance with the steps. Effects of invention

According to the present invention, superposition of a differential image map over an anatomic map comprising coordinate data which represent shapes and haptic data on, for example, the hardness and the pressure sensitivity which are contained in shape data enables indication of at which part of a body and to what extent a body shape difference between before and after surgical excision of a diseased portion and a state of hardness are generated.

Thus, numerical value information provided by the present invention facilitates adjustment of, for example, a breast shape by a doctor during reconstructive surgery, thereby enabling support for more ideal reconstructive surgery.

### BRIEF DESCRIPTION OF DRAWINGS

FIG. 1 is an overall configuration diagram according to one embodiment of the present invention.

FIG. 2 is a block configuration diagram according to one embodiment of the present invention.

FIG. 3 shows an example of an attachment part according to one embodiment of the present invention.

FIG. 4 shows an example illustrating a state of a tactile sensor when the attachment part according to one embodiment of the present invention is attached: FIG. 4(a) is a diagram illustrating a state when the attachment part is attached, the state being seen from the side; and FIG. 4(b) is a diagram in which a b portion of the attachment part in FIG. 4(a) is enlarged.

### DESCRIPTION OF EMBODIMENTS

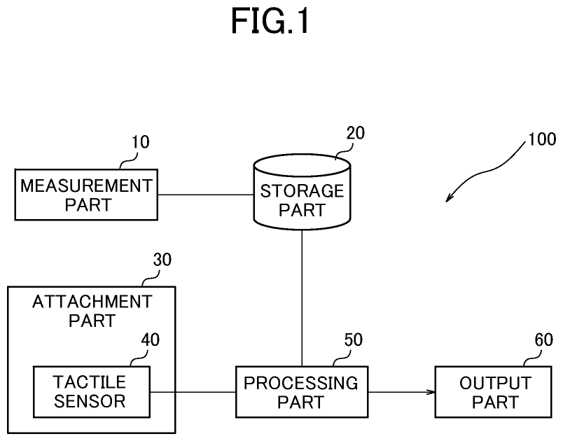

Hereinafter, one embodiment of the present invention will be described with reference to FIG. 1 to FIG. 4.

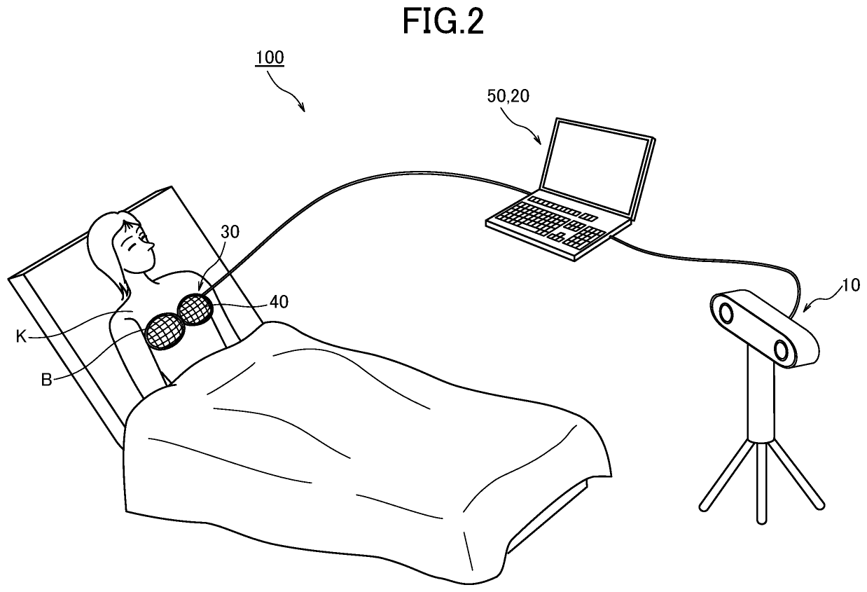

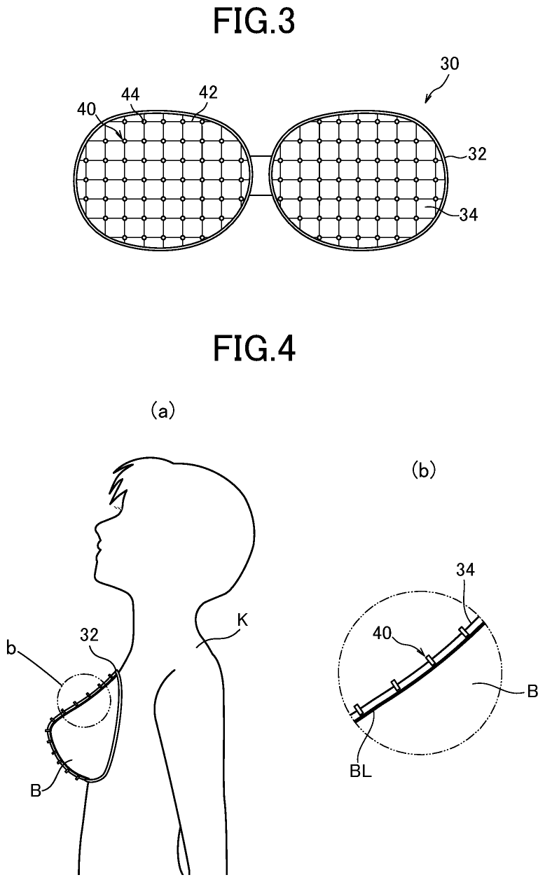

FIG. 1 is an overall configuration diagram according to one embodiment of the present invention. FIG. 2 is a block configuration diagram according to one embodiment of the present invention. FIG. 3 shows an example of an attachment part according to one embodiment of the present invention. FIG. 4 shows an example illustrating a state of a tactile sensor when the attachment part according to one embodiment of the present invention is attached: FIG. 4(a) is a diagram illustrating a state when the attachment part is attached, the state being seen from the side; and FIG. 4(b) is a diagram in which a b portion of the attachment part in FIG. 4(a) is enlarged.

In the following description, the configurations denoted by the same reference signs in different drawings are similar, and thus a description thereof may be omitted.

A body mapping device for correlating a shape of a protruding physical portion and a dynamic feature with each other and measuring the same according to the present invention may have any configuration as long as the device comprises: a measurement part which measures three-dimensionally a shape of the physical portion; a storage part which stores coordinate data of the shape acquired by the measurement part; an attachment part which is attached tightly to the physical portion and provided with a grid having a certain grid spacing; a plurality of tactile sensors which are arranged at corresponding intersections of the grid of the attachment part and measure the dynamic feature in a direction vertical to a surface of contact between the physical portion and the attachment part; and a processing part which associates haptic data acquired by the tactile sensors with the coordinate data of the intersections at which the haptic data are acquired, and creates the body map.

### Explanation of the Overall Configuration

With reference to FIG. 1 and FIG. 2, a body mapping device 100 according to one embodiment of the present invention includes an attachment part 30 which is attached tightly to a breast B that is a protruding physical portion of a measured person K, tactile sensors 40 arranged at the attachment part 30, a measurement part 10 which scans a shape of the breast B of the measured person K and acquires three-dimensional coordinate data, a storage part 20 which stores the coordinate data, and a processing part 50 which carries out information processing to associate the coordinate data with haptic data acquired by the tactile sensors 40.

Further, the body mapping device 100 may be configured to further include an output part 60 which outputs a result of the information processing carried out by the processing part 50.

The measurement part 10 measures a three-dimensional shape of an object and may be of a contact type or a non-contact type. In the present embodiment in which haptic data are acquired at the same time, the measurement part 10 of a non-contact type is preferable to prevent changes of haptic data due to contact.

A non-contact type 3D scanner is a device for sensing unevenness of an object and acquiring the same as 3D data. For example, a non-contact type 3D scanner emits laser light onto an object to acquire a plurality of sets of three-dimensional coordinate data (X, Y, Z). Such acquired “point cloud data” are converted into “polygon data” to generate a solid.

For a 3D scan, a so-called “3D body scan” for which a large number of products and services are provided (for example, by Wacoal Corp.:

https://www.wacoal.jp/smart_try/service/3d/) may be employed and no special specification is required.

The storage part 20 stores coordinate data acquired by the measurement part 10 and, for the storage part 20, a storage medium for a computer can be employed.

The attachment part 30 desirably has a shape and is made of a material so as to have a property, such as tight attachment, stretchability with the shape retention, and no oppression in relation to the protruding physical portion. For the attachment part 30, an attachment wear similar to a brassiere may be employed when the protruding physical portion is the breast B.

Further with reference to FIG. 3 and FIG. 4, the attachment part 30 may include a frame 32 which surrounds an outer circumference of the breast B as illustrated in FIG. 3 and a veil 34 a rim of which is fitted to the frame 32, the veil 34 being made of an elastic material for retaining the silhouette of the breast B as illustrated in FIG. 4(a).

The frame 32 may have a wire-like shape in which the wire is made of resin or metal and used, for example, for a brassiere.

Further, for the veil 34, for example, a stretchable resin film which is elastic as described above may be used so that more accurate shape data can be assured.

The stretchable resin film is made of a soft and flexible film-like insulating material and has an excellent expansibility, which is preferable also for arranging the tactile sensors.

As illustrated in FIG. 3, the surface of the veil 34 of the attachment part 30 is provided with a grid 42 having a certain grid spacing. As illustrated in FIG. 4(a) and FIG. 4(b), the plurality of tactile sensors 40 are arranged at corresponding intersections 44 of the grid 42 to measure a dynamic feature in a direction vertical to a surface BL of contact between the breast B and the attachment part 30.

The grid 42 is not limited to having a rectangular shape, such as a square, having an exact dimension and an equal grid spacing but may have a plurality of measurement points suitable for identifying coordinate data with haptic data at the time of body mapping and providing support at the time of reconstruction.

For the tactile sensors 40, a force sensor of the following types may be employed, as appropriate.

A tactile sensor of an electrical resistance type detects the magnitude of a force using an object in which an electrical resistance value changes in a certain manner when a force is applied.

A tactile sensor of a capacitive type detects the magnitude of a force using a configuration in which the capacitance changes in a certain manner due to a force.

A tactile sensor of a piezoelectric type detects the magnitude of a force using a piezoelectric element in which the voltage is generated when a force is applied.

A tactile sensor of an optical type in which a part at which a force is to be applied has a pattern printed detects through an optical sensor a change of the pattern due to a force to determine the magnitude of the force.

Data measured by the tactile sensors 40 is transmitted as an electrical signal to the processing part 50. Then, in order to be able to identify which intersection 44 of the grid 42, the position of the intersection 44 is also transmitted as data to the processing part 50.

The processing part 50 associates haptic data acquired by the tactile sensors 40 with the coordinate data of the intersections 44 at which the haptic data are acquired, and creates the body map.

The haptic data contain haptic data of a dynamic feature and coordinate data, and the processing part 50 correlates the coordinate data and the corresponding haptic data with each other.

The processing part 50 is formed by a micro computer and includes a processor CPU to carry out calculations, a ROM to store control programs and means necessary for calculations and recordings, such as lists, tables, and maps for various data, and a RAM to temporarily store calculation results from the CPU and the like.

The processing part 50 includes a non-volatile memory, and this non-volatile memory stores necessary data and the like. The non-volatile memory may be formed by an EEPROM which is a rewritable ROM, or a RAM with a backup function by means of which a holding current is supplied, even when the power is turned off, to retain memory.

Note that the storage part 20 may be formed as a part of a micro computer provided with the processing part 50.

For the output part 60, a typical monitor or printer may be employed. Data outputted from the output part 60 are preferably processed by being converted to a graph, a table or the like so as to be easily viewed by a doctor or the measured person K.

Note that imaging an output and coloring haptic data of corresponding coordinates according to levels can improve the convenience for grasping measurement results.

### Explanation of Uses of the Present Embodiments

### (1) Support for Reconstructive Surgery of a Protruding Physical Portion, Such as a Breast

According to the present embodiments, superposition of a differential image map over an anatomic map comprising coordinate data relating to shapes and hardness and pressure-sensitivity data contained in the coordinate data enables indication of at which part of a body and to what extent a body shape difference between before and after surgical excision of a diseased portion and a state of hardness are generated. Accordingly, numerical value information provided by the present invention facilitates adjustment of, for example, a breast shape by a doctor during reconstructive surgery, thereby enabling support for more ideal reconstructive surgery.

### (2) Support for Anomaly Detection

According to the present embodiments, comparison using a criteria map which indicates a relationship between acquired mapping data from a body map and haptic data having a statistically predetermined range allows for abnormal value determination (for example, about a cancer) and diagnosis support for information processing and evaluation of changes of coordinate data due to gravity and accompanying changes of hardness and pressure-sensitivity data, and the like.

Further, construction of a database for medical knowledge and criteria maps with diagnosis statuses so far enables determination and threshold value setting in analogy to an expert system.

### (3) Visualization Before and After Reconstructive Surgery

According to the present embodiments, visualization of a body map before and after reconstructive surgery, for example, generation of a body map colored according to levels of a pressure state as haptic data allows the measured person K to check by herself the reproducibility after reconstructive surgery.

### (4) Production of a Part for Reconstructive Surgery

According to the present embodiments, in accordance with the hardness, the pressure, the elasticity, etc. based on a shape of and a sense of touch for a part to be produced for reconstructive surgery, selection of a suitable material and design of a structure can be made in advance and a part can be produced so as to meet requests by the measured person K to undergo reconstructive surgery.

### (5) Application for Cosmetic Surgery

According to the present embodiments, statistical processing of coordinate data relating to a plurality of shapes accumulated and haptic data enables construction of a structural simulation model for a protruding physical portion, such as a breast.

The structural simulation model can be used for, for example, a preliminary consideration in the case of, for example, breast augmentation to enlarge the breast B.

Note that for a structural simulation model, a finite element method, a boundary element method, and the like can be employed.

As described above, according to the present invention, superposition of a differential image map over an anatomic map comprising coordinate data which represent shapes and haptic data on, for example, the hardness and the pressure sensitivity which are contained in shape data enables indication of at which part of a body and to what extent a body shape difference between before and after surgical excision of a diseased portion and a state of hardness are generated.

Thus, numerical value information provided by the present invention facilitates adjustment of, for example, a breast shape by a doctor during reconstructive surgery, thereby enabling more ideal reconstructive surgery.

### REFERENCE SIGNS LIST

10 measurement part
20 storage part
30 attachment part
32 frame
34 veil
40 tactile sensor
42 grid
44 intersection
50 processing part
60 output part
100 body mapping device
K measured person
B breast (protruding physical portion)
BL surface of contact

1. A body mapping device for correlating a shape of a protruding physical portion and a dynamic feature with each other and measuring the same, the body mapping device being characterized by comprising:
a measurement part which measures three-dimensionally a shape of the physical portion;
a storage part which stores coordinate data of the shape acquired by the measurement part;
an attachment part which is attached tightly to the physical portion and provided with a grid having a certain grid spacing;
a plurality of tactile sensors which are arranged at corresponding intersections of the grid of the attachment part and measure the dynamic feature in a direction vertical to a surface of contact between the physical portion and the attachment part; and
a processing part which associates haptic data acquired by the tactile sensors with the coordinate data of the intersections at which the haptic data are acquired, and creates the body map.

2. The body mapping device of claim 1, characterized in that the attachment part includes:
a frame which surrounds an outer edge of the physical portion; and
a veil a rim of which is fitted to the frame, the veil being made of an elastic material.

3. The body mapping device of claim 1, characterized in that the haptic data are force data.

4. The body mapping device according to claim 3, characterized in that the tactile sensors include a force sensor of an electrical resistance type, a capacitive type, a piezoelectric type or an optical type.

5. The body mapping device of claim 3, characterized in that the processing part
includes a criteria map which indicates a relationship between coordinate data of the physical portion of a healthy body and haptic data having a statistically predetermined range in advance, and
transmits a signal when measured haptic data do not fall within the predetermined range of the criteria map.

6. The body mapping device of claim 1, characterized in that the physical portion is a breast.

7. The body mapping device of claim 1, wherein the attachment part is attached to a breast reconstructed after breast excision by surgical excision of a diseased portion to measure the reconstructed breast on the basis of the body map as of presurgery.

8. A body mapping method for correlating a shape of a protruding physical portion and a dynamic feature with each other and measuring the same, comprising:
a step of measuring three-dimensionally a shape of the physical portion;
a step of storing coordinate data of the shape acquired by the measurement part;
a step of attaching an attachment part which is attached tightly to the physical portion and provided with a grid having a certain grid spacing; and
a step of creating the body map by associating haptic data acquired by a plurality of tactile sensors which are arranged at corresponding intersections of the grid of the attachment part and measure the dynamic feature in a direction vertical to a surface of contact between the physical portion and the attachment part with coordinate data of the intersections at which the haptic data are acquired.
